(19) United States
(12) Patent Application Publication
(10) Pub. No.: US 2009/0030426 A1
(43) Pub. Date: Jan. 29, 2009

(54) Catheter Tunneler and Method of Using Same

(75) Inventors: Kenneth M. Zinn, Westport, CT (US); Mark S. Fisher, Sellersville, PA (US)

Correspondence Address: Fox Rothschild LLP;Blue Bell, 2000 Market Street, Tenth Floor, Philadelphia, PA, 19103, US

(73) Assignees: Innovative Medical Devices, LLC, Westport, CT (US); Medical Components, Inc., Harleysville, PA (US)

(21) Appl. No.: 12/175,913

(22) Filed: Jul. 18, 2008

Related U.S. Application Data
(60) Provisional application No. 60/961,746, filed on Jul. 24, 2007.
(60) Provisional application No. 60/967,053, filed on Aug. 31, 2007.

(51) Int. Cl. A61M 25/01; A61M 25/16

(52) U.S. Cl. 606/108; 604/533

(57) ABSTRACT

A tunneler/casing assembly (100,200,300) having a tunneler (110,210,310) and an elongated casing (102,202,302) for containing a catheter (12) during subcutaneous tunneling of the catheter. The tunneler is removable from the casing to expose the catheter leading end after tunneling to be pulled directly through the tunnel, or includes a catheter exit side opening (324) to expose the catheter leading end for that purpose. A blunt leading end (112,212,312) of the tunneler has an atraumatic shape for creating the subcutaneous tunnel (26).

### CROSS-REFERENCE TO RELATED APPLICATIONS

This application claims the benefit of U.S. Provisional Application Ser. Nos. 60/967,053 filed Aug. 31, 2007 and 60/961,746 filed Jul. 24, 2007.

### FIELD OF THE INVENTION

This relates to the field of medical devices, and more particularly to tunnelers used with catheters for subcutaneous anchoring of a proximal catheter portion during implantation of the catheter into a patient's vasculature.

### BACKGROUND OF THE INVENTION

When a catheter assembly is implanted into the vasculature of a patient, the catheter's distal portion is inserted through an incision into the vasculature until the distal tip is precisely located at the desired site, while the proximal portion remains outside the vasculature. The proximal end portion remains external of the patient for access to the catheter for infusion of fluids or withdrawal thereof, or for connections with a hemodialysis apparatus. In order to assure that stress and strain on the catheter assembly do not result in movement of the distal tip from its proper location and to also protect against infection, especially with a long-term catheter, a variable length of the catheter is placed through a subcutaneous tunnel. This is accomplished by use of a tunneler or a trocar. The standard tunneler pulls the catheter after attachment to an end thereof, through the subcutaneous tissue. Tunneling may be performed either by attachment of the tunneler to the distal catheter end prior to its insertion into the vasculature, or by attachment to the proximal end of the catheter for tunneling after the distal portion has been placed in the vein, termed retrograde tunneling.

Commonly, the tunneler is a generally inflexible cylindrical shaft with a blunt tip for advancing subcutaneously between a location near the catheter's venous entranced site to a tunnel exit site, creating the tunnel. The opposite or connection end of the tunneler is first attachable to the catheter end, and it is later removable from the catheter end after tunneling. There are several known manners of catheter/tunneler attachment, including those disclosed in U.S. Pat. Nos. 4,453,928; 4,832,687; 5,190,529; 5,944,732; 6,453,185; and 6,872,198; and also in U.S. Patent Publications Nos. US 2004/0176739; US 2004/0193119; and US 2005/0027282. Other connections of devices to ends of catheters are disclosed in U.S. Pat. Nos. 5,360,407 and 5,637,102 wherein a proximal end of a catheter is inserted over a barbed locking device, with assistance from an outer locking sleeve to assure the connection.

In U.S. Publication No. US 2005/0027282, an adapter is disclosed attachable to the connection end of the tunneler, wherein an open end of the adapter permits insertion of the catheter end, such as the distal end portion, whereafter a plurality of gripping sections is moved into a gripping relationship to the outside surfaces of the catheter and locked into position; after tunneling, the gripping sections are unlocked releasing the catheter for withdrawal.

It is desired to provide a tunneler that is easily used with small-diameter catheters, such as PICCs (peripherally insertable central catheters) or certain centrally insertable catheters.

It is also desired to provide a tunneler that is removable from the catheter after tunneling without having damaged the end of the catheter adjacent the tunneler during tunneling.

### BRIEF SUMMARY OF THE INVENTION

Briefly, the present invention provides a tunneler assembly having a tunneler and an elongated casing attached to the tunneler within which the catheter can reside during the tunneling procedure or through which the catheter can be passed after the tunnel has been defined but while the casing still resides in the tunnel, whereby it is unnecessary for the catheter to be mechanically connected to the tunneling device nor disconnected therefrom. The tunneler preferably has a blunt leading tip that has rounded edges and corners and is atraumatic, with an elongated beveled surface extending rearwardly therefrom toward the casing leading end. The tunneler is preferably removed from the casing's leading end after the tunnel has been created, whereafter the catheter may be pressed forward from the trailing end of the casing until the leading end of the catheter can be grasped to be pulled further, until the portion of the catheter desired to be anchored is in position within the tunnel.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is an isometric view of a first embodiment of tunneler/casing assembly of the present invention containing a catheter and having a U-shape;

FIG. 2 is an enlarged cross-sectional view of the leading end of the assembly of FIG. 1 and showing a representation of a subcutaneous tunnel with an entrance and an exit;

FIG. 3 is an isometric view of the assembly of FIGS. 1 and 2 after tunneling has been completed and the casing has been pulled out of the tunnel while the catheter portion to be anchored remains in the tunnel;

FIG. 4 is an isometric view of a second embodiment of tunneler/casing assembly of the present invention;

FIGS. 5 to 7 are isometric, side and top views of the tunneler of FIG. 4; and

FIG. 8 is a cross-sectional view of the leading end of a third embodiment of a tunneler/casing assembly of the present invention.

### DETAILED DESCRIPTION OF THE INVENTION

In the drawings, like numerals indicate like elements throughout. Certain terminology is used herein for convenience only and is not to be taken as a limitation on the present invention. The terms “distal” and “proximal” refer, respectively, to directions closer to and away from the insertion tip of a catheter in an implantable catheter assembly. The terms “leading end” and “trailing end” refer to the end of the casing/tunneler assembly or the end of the catheter with respect to the direction of its movement through the subcutaneous tunnel, whether or not the leading end of the catheter is the distal end of the catheter. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

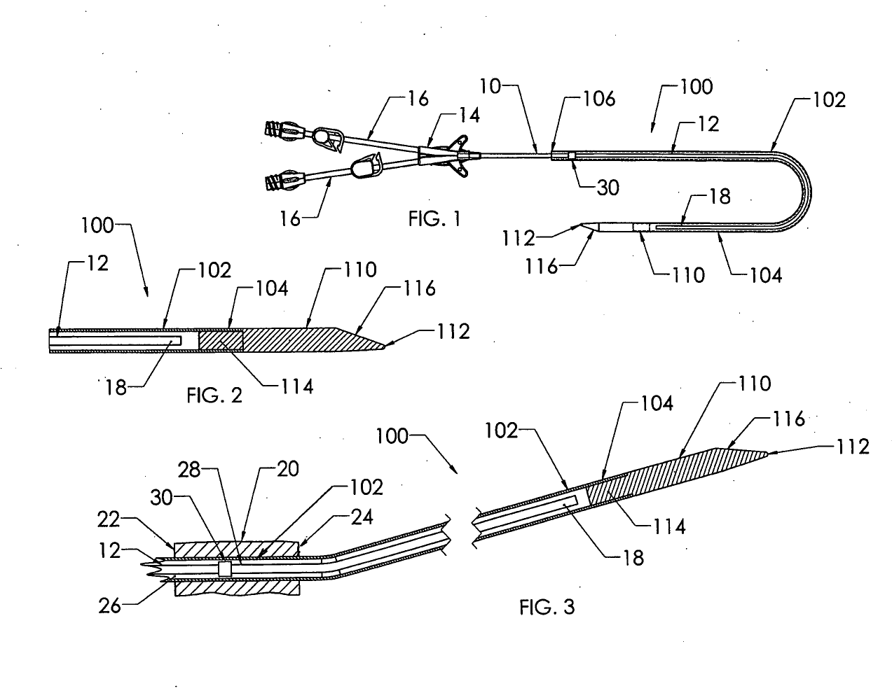

In FIGS. 1 to 3, a first embodiment of tunneler/casing assembly 100 is shown, comprising a cylindrical casing 102 having leading and trailing ends 104,106 and a tunneler 110 affixed to casing 102 at its leading end 104. Tunneler 110 includes a blunt, atraumatic tip 112 and a connection section 114 by which it is secured to casing 102, such as by being force fit into the casing leading end 104. Tunneler 110 defines an elongated beveled surface 116 extending rearwardly from blunt, rounded tip 112 toward the casing leading end, the combination of which provides its capability of separating subcutaneous tissue of the patient to define a tunnel.

In FIG. 1, a catheter assembly 10 is also shown, having a catheter 12, a hub 14, a pair of extension tube assemblies 16 (associated with respective lumens of the catheter 12) and a catheter distal end 18. Catheter 12 is shown with much of its length disposed within the casing 102, with catheter distal end 18 adjacent to the tunneler 110 at the casing leading end 104. Preferably, the catheter assembly further includes a tissue ingrowth cuff 30 which facilitates tissue ingrowth thereof within the tunnel enhancing the anchoring of the catheter. Catheter assembly 10 may be shipped already disposed within casing 102, although it need not affixed to the tunneler assembly 100; the catheter may be secured to the casing by means of a clamp at the proximal casing end, easily permitting removal of the catheter from the tunneler assembly before tunneling by the practitioner, if desired. A typical subcutaneous tunnel formed in the patient for anchoring the catheter is from about 5 cm to about 10 cm in length. A typical catheter length could be about 60 cm; the tunneler/casing assembly 100 could be, for example, about 15 cm to 20 cm. The present tunneler is especially useful with small-diameter catheters of for example 4 to 6 F.

In FIG. 1, the tunneler assembly 100 is shown to be formed into a U-shape. This shape facilitates shipping, handling and storage by reducing the over-all length, and also serves to maintain the catheter within it in a non-coiled shape, during shipping, handling and storage. The material from which the casing is manufactured preferably is sufficiently flexible to enable the practitioner to easily manipulate the casing into a linear configuration to pass through the tunnel.

Tunneler/casing assembly 100 with catheter 12 therewithin is shown in FIG. 3 after having been tunneled subcutaneously in a patient 20, from an entrance incision 22 to an exit incision 24, with tunnel 26 extending therebetween. Tunneler 110 has already been fully passed through (and thereby creating and defining) the tunnel 26, and casing 102 has been pulled substantially through tunnel 26, in turn pulling catheter leading end 18 with it. Alternatively, the tunneler/casing assembly 100 may be utilized without catheter 12 therein to create the tunnel whereafter catheter 12 may be inserted by its leading end 18 into and through the casing 102 while the casing remains within the tunnel; catheter leading end 18 may be grasped by the practitioner once tunneler 110 has been disconnected from the casing leading end 104 after tunneling, such as by being snapped off or cut off from the casing end. After the catheter assembly 10 has been tunneled and the anchor portion 28 of the catheter 12 is in position within tunnel 26 (about 5 cm to about 10 cm in length), with anchor portion 28 preferably including a conventional tissue ingrowth cuff 30. If catheter leading end 18 is the distal end of the catheter, the catheter may now be cut to length at its leading end 18 and prepared for insertion into the vasculature of the patient through a venotomy, such as with the use of a tearaway introducer sheath, dilator and guide wire (not shown), all as is conventional.

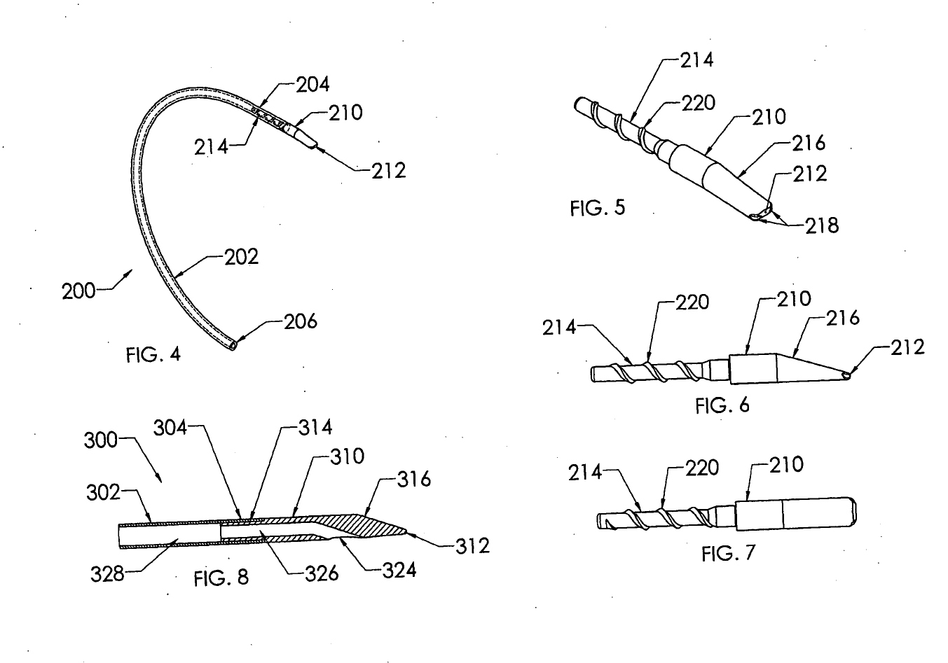

FIGS. 4 to 7 illustrate a second embodiment of tunneler/casing assembly 200 of the present invention, with a casing 202 having a leading end 204 and a trailing end 206, and a tunneler 210 having a blunt tip 212, a connection section 214 and an elongated beveled surface 216 extending to blunt tip 212. FIG. 5 especially shows that blunt tip 212 includes rounded edges and a pair of rounded corners 218, thereby being atraumatic which is preferable in all embodiments of the present invention. Connection section 214 of tunneler 210 is shown to include an exterior thread 220 so that it may be connected to casing 202 by being threaded into casing leading end 204; tunneler 210 is likewise easily disconnected and removed from the leading end of casing 202 by simply being unthreaded therefrom. Optionally, the connection section could instead include one or more barbs (not shown) instead of a thread. Casing 202 of assembly 200 may be made of the same material as the casing of the first embodiment, and is shown to have a generally curving nature that may facilitate being pushed through the subcutaneous tissue to create the tunnel.

A third embodiment of casing tunneler assembly 300 is shown in FIG. 8. Tunneler 310 includes a connection section 314 adapted to be force fit into leading end 304 of casing 302, as with the embodiment of FIGS. 1 to 3. Tunneler 310 also includes a blunt, rounded atraumatic tip 312 and tapered surface 316 extending rearwardly therefrom toward casing leading end 304. In tunneler 310 is defined a side opening 324 in communication with a central passageway 326 that extends to the hollow passageway 328 of casing 302. Side opening 324 provides the tunneler/casing assembly 300 with the option of permitting the practitioner to push the trailing end of catheter 12 into the casing and the tunnel until the catheter leading end 14 passes into passageway 326 and outwardly of the tunneler/casing assembly 300 at side opening 324 so that the leading end 18 may be grasped by the practitioner and pulled directly, through the casing and the tunnel, whereafter the tunneler/casing assembly can then be removed from the catheter and the tunnel, and discarded.

The tunneler of the present invention may be molded of plastic, such as for example polypropylene or a nylon 6/ABS blend. The casing of the present invention may be extruded of plastic, such as for example, polyethylene. The casing preferably is longer than the desired length of the tunnel, and preferably is greater than 10 cm, and more preferably is greater than 15 cm. An increased length of the casing results in greater assurance that the catheter is self retaining within the casing during tunneling. The overall length of the casing may be reduced prior to tunneling, by the practitioner simply trimming excess from the proximal end thereof.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A tunneling device for subcutaneous tunneling of a vascular catheter, comprising:
an elongated cylindrical casing having a leading end and a trailing end, and a tunneler removably affixed to the casing at the casing leading end and extending therefrom, the tunneler having a blunt atraumatic leading tip and an elongated beveled surface extending toward the casing leading end.

2. The tunneling device of claim 1, wherein the tunneler includes a connection section extending into the casing leading end.

3. The tunneling device of claim 2, wherein the connection section is held by force fit within the casing leading end.

4. The tunneling device of claim 2, wherein the connection section is threaded and is held within the casing leading end by being threaded thereinto.

5. The tunneling device of claim 2, wherein the tunneler includes a passageway through the connection section and extending to a side opening.

6. The tunneling device of claim 1, wherein the tunneler/casing assembly is formed in a U-shape of generally flexible material.

7. The tunneling device of claim 1, wherein the tunneler/casing assembly is at least 10 cm in length.

8. The tunneling device of claim 1, wherein the tunneler/casing assembly is at least 15 cm in length.

9. An assembly of a catheter assembly and a tunneling device for subcutaneous tunneling of the catheter of the catheter assembly, comprising:
a catheter assembly having a catheter with a catheter leading end extending toward a catheter trailing end, and
an elongated cylindrical casing having a leading end and a trailing end, and a tunneler removably affixed to the casing at the casing leading end and extending therefrom, the tunneler having a blunt atraumatic leading tip and an elongated beveled surface extending toward the casing leading end,
the catheter being disposed within the casing such that the catheter leading end is adjacent the tunneler and the trailing end of the catheter extends from and outwardly of the trailing end of the casing, and the inner diameter of the casing is selected such that the catheter is held by a slight friction fit within the elongated casing so that the catheter remains within the casing during creation of the subcutaneous tunnel by the tunneler, whereafter the tunneler/casing assembly and the catheter are pulled through the tunnel, and the tunneler/casing assembly is removable from the catheter at the catheter leading end.

10. The assembly of claim 9, wherein the tunneler includes a connection section extending into the casing leading end.

11. The assembly of claim 10, wherein the connection section is held by force fit within the casing leading end.

12. The assembly of claim 10, wherein the connection section is threaded and is held within the casing leading end by being threaded thereinto.

13. The assembly of claim 10, wherein the tunneler includes a passageway through the connection section and extending to a side opening.

14. The assembly of claim 9, wherein the tunneler/casing assembly is at least 10 cm in length.

15. The assembly of claim 9, wherein the tunneler/casing assembly is at least 15 cm in length.

16. A method of tunneling a catheter during implanting into a patient, comprising the steps of:
providing a casing having a passage sized to receive a catheter therethrough;
providing a tunneler affixed to a leading end of the casing and having a blunt, atraumatic tip for creating a subcutaneous tunnel in a patient the tunneler and casing defining a tunneling assembly;
inserting a catheter into a trailing end of the casing of the tunneling assembly;
pushing the tunneler through subcutaneous tissue of the patient between a tunnel entrance incision and a tunnel exit incision;
exposing a leading end of the catheter at the leading end of the casing;
pulling the catheter through the casing within the tunnel for a selected distance; and
removing the casing from the tunnel and the catheter.

17. The method of claim 16, wherein the step of exposing the catheter leading end comprises the step of removing the tunneler from the casing leading end, and pushing the catheter sufficiently for its leading end to be grasped.

18. The method of claim 16, wherein the tunneler has a threaded proximal end section to be threaded into the casing leading end, and the step of removing the tunneler from the casing leading end comprises the step of unthreading the tunneler therefrom.

19. The method of claim 16, wherein the tunneler has an angled side port adjacent the blunt tip and in communication with the passage, and the step of exposing the catheter leading end comprises the step of pushing the catheter further through the casing passage to exit the angled side port sufficiently to enable the catheter leading end to be grasped for pulling.
